(19) United States
(12) Patent Application Publication
(10) Pub. No.: US 2026/0325693 A1
(43) Pub. Date: Oct. 22, 2026

(54) CLOTHES CARE APPARATUS AND CONTROL METHOD THEREOF

(71) Applicant: SAMSUNG ELECTRONICS CO., LTD., Suwon-si (KR)

(72) Inventor: Hochae LEE, Suwon-si (KR)

(73) Assignee: SAMSUNG ELECTRONICS CO., LTD., Suwon-si (KR)

(21) Appl. No.: 19/692,758

(22) Filed: May 29, 2026

Related U.S. Application Data
(63) Continuation of application No. PCT/KR2024/015568, filed on Oct. 15, 2024.

(30) Foreign Application Priority Data
Nov. 29, 2023 (KR) 10-2023-0170179

(51) Int. Cl. A61L 2/14; A61L 2/025; A61L 2/07; A61L 2/24; A61L 103/50; D06F 58/10; D06F 87/00; D06F 103/38; D06F 103/54; D06F 105/30

(52) U.S. Cl. CPC A61L 2/14; A61L 2/025; A61L 2/07; A61L 2/24; D06F 58/10; D06F 87/00; A61L 2103/50; A61L 2202/11; A61L 2202/122; A61L 2202/14; A61L 2202/15; D06F 2103/38; D06F 2103/54; D06F 2105/30

(57) ABSTRACT

A clothes care apparatus includes: a chamber configured to accommodate clothes; a plasma supply unit configured to spray plasma into the chamber; at least one sensor configured to obtain information regarding contamination of the clothes in a state that the clothes are accommodated in the chamber; and at least one cleaning device configured to remove the contamination, and the information regarding the contamination includes information regarding a target area at which the contamination is located on the clothes, and the at least one cleaning device includes: an ultrasonic irradiator configured to irradiate ultrasonic waves onto the target area, an ultrasonic receiver configured to receive echo ultrasonic waves reflected from the target area, a fan configured to blow air to the target area, and a fan motor configured to generate a rotation force of the fan.

### CROSS-REFERENCE TO RELATED APPLICATIONS

This application is a continuation of International Application No. PCT/KR2024/015568, filed on Oct. 15, 2024, which is based on and claims priority to Korean Patent Application No. 10-2023-0170179, filed on Nov. 29, 2023, in the Korean Intellectual Property Office, the disclosures of which are incorporated by reference herein in their entireties.

### BACKGROUND

### 1. Field

The present disclosure relates to a clothes care apparatus and a control method thereof.

### 2. Description of Related Art

A clothes care apparatus is equipment for clothes care, such as drying wet clothes, removing dust gathered on clothes or smell permeated in clothes, and smoothing out the wrinkles of clothes.

In general, the clothes care apparatus includes a machine room that contains a steam generator or a hot water supply device, etc. to perform a refresh function, such as removing the wrinkles of clothes and the like, deodorization, removal of static electricity, etc.

The clothes care apparatus may perform a cleaning process to remove contamination from clothes contained therein. The cleaning process may include a process of spraying plasma humidified air or detergent onto clothes contained within the clothes care apparatus.

When plasma is sprayed onto clothes, low-velocity plasma is sprayed onto the clothes, and thus methods for enhancing the physical force acting on the clothes are actively being researched.

### SUMMARY

According to an aspect of the disclosure, there is provided a clothes care apparatus including: a chamber configured to accommodate clothes; a plasma supply unit configured to spray plasma into the chamber; at least one sensor configured to obtain information regarding contamination of the clothes in a state that the clothes are accommodated in the chamber; and at least one cleaning device configured to remove the contamination, and the information regarding the contamination includes information regarding a target area at which the contamination is located on the clothes, and the at least one cleaning device includes: an ultrasonic irradiator configured to irradiate ultrasonic waves onto the target area, an ultrasonic receiver configured to receive echo ultrasonic waves reflected from the target area, a fan configured to blow air to the target area, and a fan motor configured to generate a rotation force of the fan.

The clothes care apparatus may include: at least one focusing device configured to move the at least one cleaning device inside the chamber; and a controller comprising a processor, the controller being operatively connected to the plasma supply unit, the at least one sensor, the at least one cleaning device, and the at least one focusing device, wherein the controller is configured to, based on the information regarding the target area, control the at least one focusing device to move the at least one cleaning device to a location at which the at least one cleaning device is capable of irradiating the ultrasonic waves onto the target area.

The controller may be further configured to, based on a speed of the echo ultrasonic waves received by the ultrasonic receiver being equal to or greater than a first reference value, turn on the fan.

The controller may be further configured to: determine, based on a time length from a first time at which the ultrasonic irradiator irradiates the ultrasonic waves until a second time at which the ultrasonic receiver receives the echo ultrasonic waves, a distance from the at least one cleaning device to the contamination, and turn on the fan within a reference distance range from the contamination.

The at least one cleaning device may further include a wind direction controller coupled to the fan motor and configured to adjust a blowing direction of the fan, and the controller may be further configured to determine a control parameter of the fan according to the speed of the echo ultrasonic waves received by the ultrasonic receiver.

The control parameter of the fan may include at least one of Revolutions Per Minute (RPM) of the fan motor, a driving time, or a rotation speed of the wind direction controller.

The controller may be further configured to, based on the speed of the echo ultrasonic waves received by the ultrasonic receiver being less than the first reference value, turn off the fan.

The clothes care apparatus may include an air spray unit configured to supply outside air into the chamber, and the controller may be further configured to control the air spray unit based on a speed of the echo ultrasonic waves received by the ultrasonic receiver.

The controller may be further configured to, based on the speed of the echo ultrasonic waves received by the ultrasonic receiver being less than a second reference value that is equal to or greater than a first reference value, turn off the air spray unit and turn on the fan.

The controller may be further configured to, based on the speed of the echo ultrasonic waves received by the ultrasonic receiver being equal to or greater than a second reference value, turn on the air spray unit and the fan.

The controller may be further configured to, based on the speed of the echo ultrasonic waves received by the ultrasonic receiver being equal to or greater than the second reference value, determine a control parameter of the air spray unit.

The control parameter of the air spray unit may include at least one of a spray amount or a spray time.

The controller may be further configured to, based on the speed of the echo ultrasonic waves received by the ultrasonic receiver being less than the second reference value, turn off the air spray unit.

According to an aspect of the disclosure, there is provided a control method of a clothes care apparatus, including: obtaining information regarding a target area at which contamination is located on clothes accommodated in a chamber; moving, based on the information regarding the target area, at least one cleaning device, configured to remove the contamination, to a position at which the at least one cleaning device is capable of irradiating ultrasonic waves onto the target area; controlling the at least one cleaning device to irradiate the ultrasonic waves onto the target area; receiving echo ultrasonic waves reflected from the target area; and controlling, based on a speed of the echo ultrasonic waves, a fan of the at least one cleaning device.

The controlling of the fan based on the speed of the echo ultrasonic waves may include, based on the speed of the echo ultrasonic waves being equal to or greater than a first reference value, turning on the fan.

The control method may further include controlling an air spray unit of the clothes care apparatus based on the speed of the echo ultrasonic waves.

The controlling of the fan may include, based on the speed of the echo ultrasonic waves being less than a second reference value that is equal to or greater than the first reference value, turning on the fan.

The controlling the air spray unit may include, based on the speed of the echo ultrasonic waves being less than the second reference value, turning off the air spray unit.

The controlling of the fan may include, based on the speed of the echo ultrasonic waves received by the ultrasonic receiver being equal to or greater than the second reference value, turning on the fan.

The controlling of the air spray unit may include, based on the speed of the echo ultrasonic waves received by the ultrasonic receiver being equal to or greater than the second reference value, turning on the air spray unit.

According to an aspect of the disclosure, a clothes care apparatus may maintain a temperature of clothes on which contamination is located within a preset range to increase efficiency of removing the contamination from the clothes by applying an indirect physical force.

According to an aspect of the disclosure, a clothes care apparatus may minimize damage to clothes while indirectly applying a physical force to the clothes in a cleaning operation using plasma.

The technical aspects achieved by the present disclosure are not limited to the above-mentioned technical objects, and other technical aspects not mentioned will be clearly understood by one of ordinary skill in the technical art to which the present disclosure belongs from the following description.

### BRIEF DESCRIPTION OF THE DRAWINGS

The above and other aspects, features, and advantages of specific embodiments of the present disclosure will be more apparent from the following description with reference to the accompanying drawings, in which:

FIG. 1 is a perspective view of a clothes care apparatus according to an embodiment;

FIG. 2 shows a state in which a door of a clothes care apparatus according to an embodiment opens;

FIG. 3 is a cross-sectional view of a clothes care apparatus according to an embodiment;

FIG. 4 shows a cleaning device according to an embodiment;

FIG. 5 shows a cleaning device according to another embodiment;

FIG. 6 is a diagram for describing how a fan included in a cleaning device according to an embodiment blows air onto a contaminated area of clothes;

FIG. 7 is a control block diagram of a clothes care apparatus according to an embodiment;

FIG. 8 is a diagram for describing how a cleaning device according to an embodiment irradiates ultrasonic waves onto contamination;

FIG. 9 is a diagram for describing how a cleaning device according to an embodiment receives echo ultrasonic waves from contamination and detects a temperature of a contaminated area of clothes;

FIG. 10 shows an example in which a clothes care apparatus according to an embodiment includes a plurality of cleaning devices;

FIG. 11 shows an example in which a clothes care apparatus according to an embodiment includes a plurality of chambers and a cleaning device included in each chamber;

FIG. 12 is a flowchart illustrating a cleaning operation according to an embodiment;

FIG. 13 is a flowchart illustrating controlling a fan included in a cleaning device in a cooling operation of FIG. 12 according to an embodiment; and

FIG. 14 is a flowchart illustrating controlling an air spray unit included in the cleaning device in the cooling operation of FIG. 12 according to an embodiment.

### DETAILED DESCRIPTION

Various embodiments of the present document and terms used therein are not intended to limit the technical features described in this document to specific embodiments, and should be understood to include various modifications, equivalents, or substitutes of the corresponding embodiments.

In connection with the description of the drawings, similar reference numerals may be used for similar or related components.

The singular form of a noun corresponding to an item may include one or a plurality of the items unless clearly indicated otherwise in a related context.

In this document, phrases, such as “A or B”, “at least one of A and B”, “at least one of A or B,” “A, B or C,” “at least one of A, B and C,” and “at least one of A, B, or C”, may include any one or all possible combinations of items listed together in the corresponding phrase among the phrases. For example, the expression, “at least one of A, B, or C,” should be understood as including only A, only B, only C, both A and B, both A and C, both B and C, or all of A, B, and C.

As used herein, the term “and/or” includes any and all combinations of one or more of associated listed items.

Terms such as “1^{st}”, “2^{nd}”, or “first” or “second” may be used simply to distinguish a component from other components, without limiting the component in other aspects (e.g., importance or order).

Some (e.g., a first) component being referred to as “coupled” or “connected”, with or without the terms “functionally” or “communicatively”, to another (e.g., second) component means that any of the above components can be connected to the other components directly (e.g., by wire), wirelessly, or via a third component.

It will be understood that the terms “includes,” “comprises,” “including,” and/or “comprising,” when used in this specification, specify the presence of stated features, figures, steps, operations, components, members, or combinations thereof, but do not preclude the presence or addition of one or more other features, figures, steps, operations, components, members, or combinations thereof.

It will be understood that when a certain component is referred to as being “connected to”, “coupled to”, “supported by” or “in contact with” another component, it can be directly or indirectly connected to, coupled to, supported by, or in contact with the other component. When a component is indirectly connected to, coupled to, supported by, or in contact with another component, the component may be connected to, coupled to, supported by, or in contact with the other component through a third component.

It will also be understood that when a component is referred to as being “on” or “over” another component, the component can be directly on the other component or intervening components may also be present.

In the following description, the terms related to directions, such as “front”, “rear”, “left”, “right”, “upper”, and “lower” are defined based on the drawings, and the shapes and positions of the components are not limited by the terms.

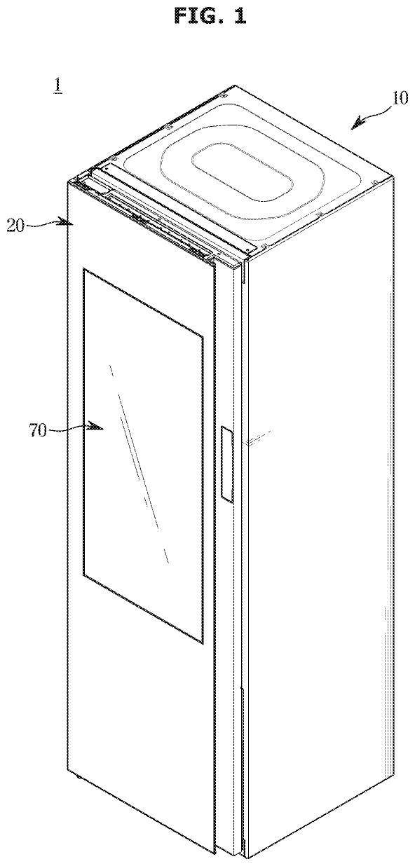

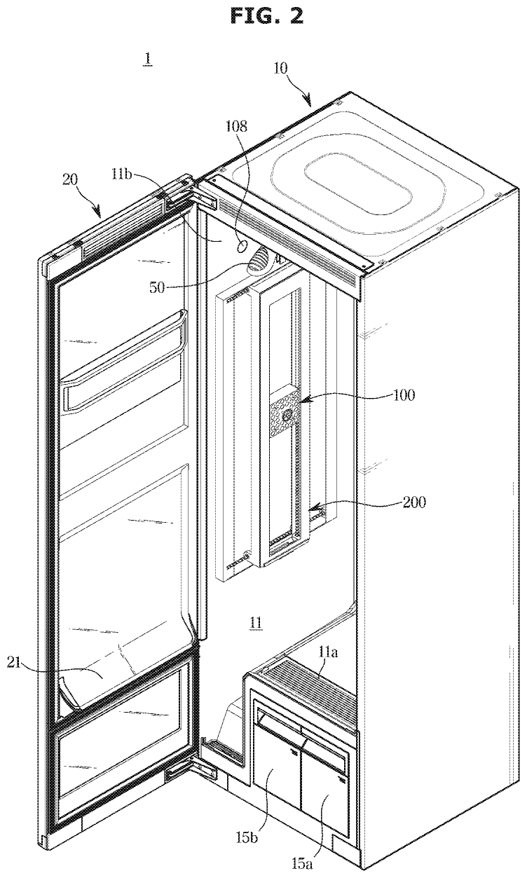

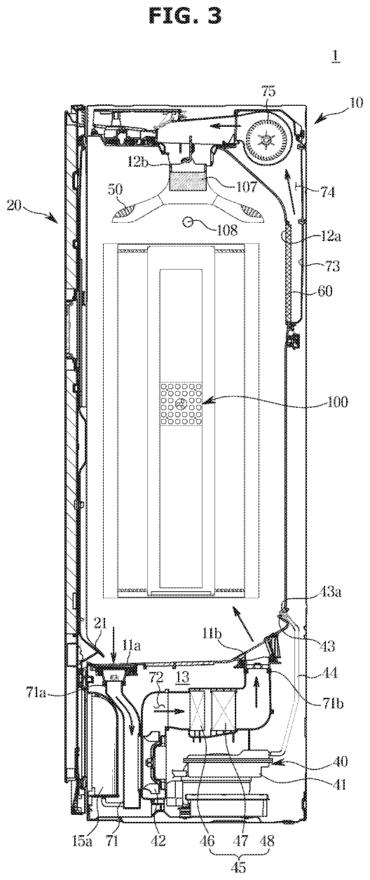

FIG. 1 is a perspective view of a clothes care apparatus according to an embodiment. FIG. 2 shows a state in which a door of a clothes care apparatus according to an embodiment opens. FIG. 3 is a cross-sectional view of a clothes care apparatus according to an embodiment.

As shown in FIGS. 1, 2, and 3, a clothes care apparatus 1 may include a cabinet 10, forming an appearance, and a door 20 rotatably coupled to the cabinet 10.

The cabinet 10 may form a chamber 11 that accommodates clothes. The cabinet 10 may have a rectangular parallelepiped shape of which one side opens. An opening may be formed at a front side of the cabinet 10. Accordingly, the chamber 11 may be in a shape of which a front side opens.

The door 20 may open or close the opening of the cabinet 10. The door 20 may be rotatably coupled to the cabinet 10. The door 20 may open or close the chamber 11 by opening or closing the opening of the cabinet 10.

A machine room 13 partitioned from the chamber 11 may be formed inside the cabinet 10. The machine room 13 may accommodate a heat exchanger 45 that heats or dehumidifies air inside the chamber 11. The machine room 13 may accommodate a steam generator 40 that supplies steam into the chamber 11.

The chamber 11 may accommodate clothes. The chamber 11 may form a space where clothes are accommodated. A clothes support member 50 for supporting clothes may be provided inside the chamber 11.

The clothes support member 50 may be detachably mounted to an upper side of the chamber 11. One or more support members 50 may be provided inside the chamber 11. For example, a plurality of support members 50 may be provided inside the chamber 11.

The clothes support member 50 may be in a shape of a hanger for hanging clothes. Air may flow inside the clothes support member 50. Air supplied to inside of the clothes support member 50 may be sprayed onto clothes, thereby removing dust or foreign materials gathered on the clothes.

The chamber 11 may include a first airflow inlet 11a, a second airflow inlet 12a, a first airflow outlet 11b, and a second airflow outlet 12b.

The first airflow inlet 11a and the first airflow outlet 11b may be formed in a lower side of the chamber 11. The first airflow inlet 11a may be positioned at a front portion of the lower side of the chamber 11. The first airflow outlet 11b may be positioned at a rear portion of the lower side of the chamber 11.

The second airflow inlet 12a may be formed in a rear side of the chamber 11. The second airflow outlet 12b may be formed in the upper side of the chamber 11. The second airflow inlet 12a may be adjacent to the second airflow outlet 12b. The second airflow inlet 12a may be provided at an upper portion of the rear side of the chamber 11. The second airflow outlet 12b may be provided adjacent to a center of the upper side of the chamber 11.

The first airflow inlet 11a may be provided with a grille portion to prevent foreign materials except for water from passing through the first airflow inlet 11a. The first airflow outlet 11b may be provided with a grille portion to prevent foreign materials from passing through the first airflow outlet 11b and cause airflow to uniformly disperse into the chamber 11.

The second airflow outlet 12b of the chamber 11 may be connected to the clothes support member 50. Air discharged through the second airflow outlet 12b may be transferred to clothes hanging on the clothes support member 50 through an air hole formed in the clothes support member 50. The clothes support member 50 may spray air that flows into the inside of the clothes support member 50 through the second airflow outlet 12b onto the clothes through the air hole. Because an airflow rate of the air sprayed through the air hole of the clothes support member 50 is very fast, dust and foreign substances adhered to the clothes may be removed.

In a lower portion of the cabinet 10, a cabinet drain container 15a and a cabinet water supply container 15b may be installed in such a way as to be separable from the cabinet 10. The cabinet drain container 15a and the cabinet water supply container 15b may be positioned below the chamber 11.

The cabinet drain container 15a may store condensed water generated while the heat exchanger 45 operates or water generated as a result of condensation of steam before the water is discharged. The cabinet drain container 15a may be separable from the cabinet 10 to easily discharge water.

The cabinet water supply container 15b may store water required to generate steam in the steam generator 40 which will be described below. The water stored in the cabinet water supply container 15b may be supplied to the steam generator 40 to be used to form steam. The cabinet water supply container 15b may be separable from the cabinet 10 to easily add water.

The cabinet drain container 15a and the cabinet water supply container 15b may be positioned in a front space of the machine room 13. The machine room 13 may be provided in the lower portion of the cabinet 10. The machine room 13 may be positioned below the chamber 11 and form a separate space from the chamber 11. The heat exchanger 45 for dehumidifying and heating inside air of the chamber 11 may be accommodated inside the machine room 13.

A first circulator 42, the heat exchanger 45, and the steam generator 40 may be positioned inside the machine room 13.

The heat exchanger 45 may supply hot dry air into the chamber 11. The heat exchanger 45 may include an evaporator 46, a compressor 48, and a condenser 47 along which a refrigerant circulates. The heat exchanger 45 may dehumidify and heat air.

While a refrigerant is evaporated in the evaporator 46 of the heat exchanger 45, the refrigerant may absorb latent heat from surrounding air, and thereby, the heat exchanger 45 may condense and remove moisture in the air. While the refrigerant is condensed in the condenser 47 via the compressor 48, the refrigerant may emit latent heat toward surrounding air, and thereby, the heat exchanger 45 may heat the surrounding air. Air having flowed into the machine room 13 by the first circulator 42 may be dehumidified and heated by sequentially passing through the evaporator 46 and the condenser 47.

The clothes care apparatus 1 may include a first duct 71 that accommodates the evaporator 46, the condenser 47, and the first circulator 42. The first duct 71 may be positioned in the machine room 13. One end 71a of the first duct 71 may be connected to the first airflow inlet 11a. Another end 71b of the first duct 71 may be connected to the first airflow outlet 11b. The first duct 71 may be connected to the chamber 11 through the first airflow inlet 11a and the first airflow outlet 11b.

Air of the chamber 11 may flow into the first duct 71 through the first airflow inlet 11a and then be discharged to the chamber 11 through the first airflow outlet 11b. Air having flowed into the first duct 71 may be dehumidified and heated by passing through the evaporator 46 and the condenser 47. The first duct 71 may form a first circulation flow path 72 along which air circulates through the chamber 11, the first circulator 42, the evaporator 46, and the condenser 47. The first circulator 42 may be positioned on the first circulation flow path 72 to suck air of the chamber 11 into the first duct 71. Air having sucked into the first duct 71 by the first circulator 42 may be discharged to the chamber 11 through the first airflow outlet 11b. The first circulation flow path 72 may be a circulation flow path through which air having flowed into the first duct 71 from the chamber 11 through the first airflow inlet 11a is again discharged to the chamber 11 from the first duct 71 through the first airflow outlet 11b.

The clothes care apparatus 1 may include the steam generator 40 positioned in the machine room 13. The steam generator 40 may generate steam by receiving water from the cabinet water supply container 15b of the machine room 13.

The steam generator 40 may include a steam generating portion 41 connected to the cabinet water supply container 15b to generate steam by receiving water, a steam spray 43 for spraying steam generated in the steam generating portion 41, and a steam connection tube 44 connecting the steam generating portion 41 to the steam spray 43.

The steam spray 43 may be fixed to a steam outlet 43a formed at the rear, lower portion of the chamber 11. The steam outlet 43a may be formed to correspond to the steam spray 43. The steam spray 43 may spray steam generated inside the machine room 13 into the chamber 11 through the steam outlet 43a. The steam spray 43 and the steam outlet 43a may be positioned at different locations.

The steam generating portion 41 may include a heater therein. By heating water through the heater of the steam generating portion 41, the steam generating portion 41 may generate steam.

The door 20 may include a door guide 21 that guides water moving downward by gravity to the first airflow inlet 11a. The door guide 21 may be provided in a lower portion of a rear surface of the first door 20. The door guide 21 may be inclined downward toward the first airflow inlet 11a from the rear surface of the door 20. Water on the rear surface of the door 20 may move to the door guide 21 by gravity and be guided in a rear direction by the door guide 21 to flow into the first airflow inlet 11a. The water having flowed into the first airflow inlet 11a may move to the cabinet drain container 15a through a connecting member.

The clothes care apparatus 1 may include a second circulator 75 for moving inside air of the chamber 11, and a second duct 73 in which the second circulator 75 is installed. The second duct 73 may be connected to the chamber 11. One end of the second duct 73 may be connected to the second airflow inlet 12a. Another end of the second duct 73 may be connected to the second airflow outlet 12b. Air having flowed into the second duct 73 through the second airflow inlet 12a may be discharged to the chamber 11 through the second airflow outlet 12b. The second circulator 75 may be installed inside the second duct 73 to suck air into the second duct 73 through the second airflow inlet 12a. Air having flowed into the second duct 73 by the second circulator 75 may be discharged to the chamber 11 through the second airflow outlet 12b.

The second duct 73 may form a second circulation flow path 74 by connecting the chamber 11, the second airflow inlet 12a, and the second airflow outlet 12b to each other. The second circulation flow path 74 may be a circulation flow path through which air having flowed into the second duct 73 from the chamber 11 through the second airflow inlet 12a is discharged to the chamber 11 from the second duct 73 through the second airflow outlet 12b.

The second duct 73 may be positioned behind the chamber 11. The second duct 73 may be positioned behind the second airflow inlet 12a formed in the rear side of the chamber 11. The second duct 73 may be positioned at an upper portion of the rear side of the chamber 11. A filter member 60 for filtering foreign materials from air flowing into the second airflow inlet 12a may be detachably installed in the second airflow inlet 12a. The filter member 60 may remove foreign materials such as dust and odors from the air flowing into the second duct 73. The filter member 60 may be provided with not only a filter but also a sheet for providing scent to clothes.

The clothes care apparatus 1 may perform a clothes care mode after clothes is hung on the clothes support member 50 and the door 20 is closed. In the clothes care mode, air inside the chamber 11 may circulate inside the cabinet 10 along the first circulation flow path 72 and the second circulation flow path 74.

The clothes care apparatus 1 may include a plasma supply unit 108 that performs a cleaning operation of spraying plasma onto clothes accommodated in the chamber 11 to remove contamination.

The plasma supply unit 108 may include a plasma generator that sucks in outside air and generates plasma, and a plasma nozzle connected to the chamber 11 to supply plasma to clothes.

The plasma supply unit 108 may be connected to the clothes support member 50 and spray plasma onto clothes while air is supplied to the inside of the clothes support member 50 and thus sprayed toward the clothes.

Also, the plasma supply unit 108 may be provided on the upper, lower, left, right, and/or rear side of the chamber 11.

A position of the plasma supply unit 108 is not limited thereto and the plasma supply unit 108 may be located at any position at which the plasma supply unit 108 is capable of efficiently removing contamination of clothes accommodated in the chamber 11. For example, the plasma supply unit 108 may be provided as a component of a cleaning device 100 that applies an indirect mechanical force to clothes in a cleaning operation, which will be described below.

The clothes care apparatus 1 may include at least one sensor 107 that obtains information on a contaminated area of clothes accommodated in the chamber 11.

The sensor 107 may be provided on an inner side 11c of the chamber 11. In a case where a plurality of sensors 107 are provided inside the chamber 11, the sensors 107 may be respectively positioned on different sides among the upper, lower, left, right, or rear side. For example, the sensor 107 may include, but is not limited to, an optical vision sensor including a camera, an ultrasonic sensor, an electrical conductivity sensor, and an infrared sensor. However, the sensor 107 may be provided as various components capable of obtaining information on a contaminated area of clothes.

The clothes care apparatus 1 may include the cleaning device 100 provided inside the chamber 11 to spot-clean clothes accommodated in the chamber 11.

The cleaning device 100 may be provided on one side wall of the chamber 11.

The clothes care apparatus 1 may move the cleaning device 100 inside the chamber 11 such that the cleaning device 100 is positioned to focus on a contaminated area of clothes.

The clothes care apparatus 1 may include a focusing device 200 that moves the cleaning device 100 inside the chamber 11.

For example, the clothes care apparatus 1 may include the focusing device 200 that moves the cleaning device 100 along a first direction and a second direction being perpendicular to the first direction on one side wall of the chamber 11, wherein the first direction may indicate a vertical direction and the second direction may indicate a horizontal direction. The vertical direction may include an up-down direction. The horizontal direction may include a front-rear direction.

As described above, the cleaning device 100 may be movable inside the chamber 11, and more specifically, the cleaning device 100 may be movable along the first direction or the second direction on one side wall of the chamber 11 by the focusing device 200.

Because the cleaning device 100 is movable in the first direction or the second direction on one side wall of the chamber 11, the cleaning device 100 may focus on a contaminated area of clothes accommodated in the chamber 11, and therefore, a ‘spot cleaning operation’ may be performed. Here, spot cleaning may mean cleaning a selected area of clothes. An area of clothes subject to spot cleaning may be referred to as a ‘target area’. The target area may be determined based on data obtained by the sensor 107.

The clothes care apparatus 1 may further include a detergent spray unit 109 that sprays a cleaning liquid onto a target area of clothes to increase cleaning power while a spot cleaning operation is performed, which is not shown. The cleaning liquid may be a mixed liquid of water and detergent.

The detergent spray unit 109 may be provided as one component of the cleaning device 100. However, a position of the detergent spray unit 109 is not limited thereto and the detergent spray unit 109 may be located at any position capable of increasing cleaning power.

The cleaning device 100 may provide micro-vibrations to clothes after or while spraying a cleaning liquid onto a target area through the detergent spray unit 109. The cleaning device 100 may provide micro-vibrations to the target area of the clothes onto which the cleaning liquid has been sprayed, thereby performing a cleaning operation on the target area.

Also, the cleaning device 100 of the clothes care apparatus 1 may further include a cleaning liquid suction unit that sucks in a cleaning liquid sprayed toward clothes. The cleaning device 100 may reduce or eliminate the cleaning liquid remaining on the clothes by sucking in the cleaning liquid. The cleaning device 100 may also absorb contaminants or foreign substances on the clothes while sucking in the cleaning liquid.

The cleaning liquid suction unit may also be provided as one component of the cleaning device 100. However, a position of the cleaning liquid suction unit is not limited thereto and the cleaning liquid suction unit may be located at any position capable of increasing cleaning power.

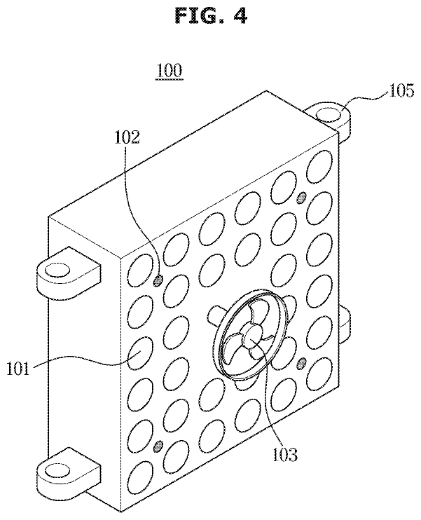

FIG. 4 shows a cleaning device according to an embodiment.

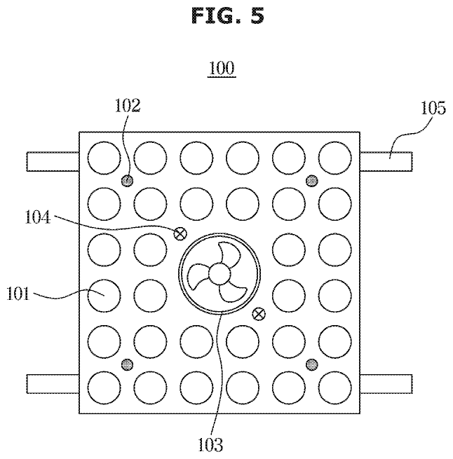

FIG. 5 shows a cleaning device according to another embodiment at another angle.

Referring to FIGS. 4 and 5, the cleaning device 100 according to an embodiment may include an ultrasonic irradiator 101 that irradiates ultrasonic waves onto contamination, an ultrasonic receiver 102 that receives echo ultrasonic waves reflected from contamination, a fan 103 that blows air to contamination, and/or a moving portion 105 connected to the focusing device 200.

The ultrasonic irradiator 101 may indirectly provide a mechanical force without directly contacting a target area of clothes by irradiating ultrasonic waves toward the contamination to apply physical pressure. The ultrasonic irradiator 101 may be implemented in a form of an Airborne Ultrasound Phased Array (AUPA).

The ultrasonic irradiator 101 may include a plurality of ultrasonic generators that generate ultrasonic waves. The ultrasonic generators may include piezoelectric elements to convert electrical energy into mechanical energy (vibration). The piezoelectric elements may have various physical properties and structures. By adjusting the physical properties and structures of the piezoelectric elements, a frequency of generated ultrasonic waves may be adjusted.

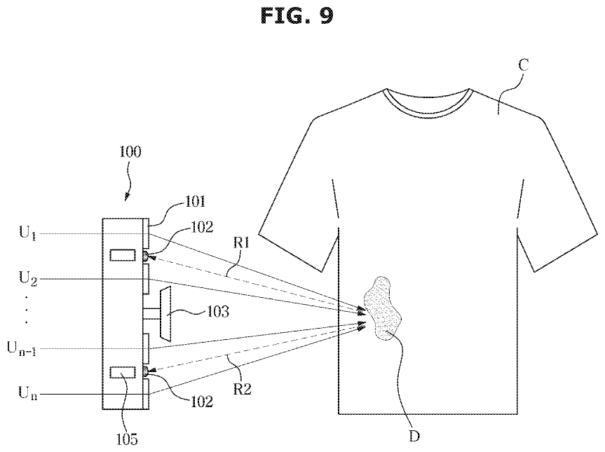

Also, the ultrasonic irradiator 101 may include a beam former that adjusts a direction of ultrasonic waves generated by the plurality of ultrasonic generators and focuses the generated ultrasonic waves on a desired focal point. Accordingly, the ultrasonic irradiator 101 may set a focal point to correspond to contamination detected by the sensor 107 and irradiate focused ultrasonic waves onto the contamination. This will be described in detail with reference to FIG. 9, below.

The ultrasonic receiver 102 may receive echo ultrasonic waves formed when at least a part of ultrasonic waves irradiated toward contamination is reflected.

When the ultrasonic irradiator 101 irradiates ultrasonic waves onto contamination, a part of the irradiated ultrasonic waves may be propagated through a target (for example, a target area of clothes accommodated inside the main body 11) and another part may be reflected from the target and then return to the cleaning device 100. At this time, the ultrasonic receiver 102 may receive the reflected ultrasonic waves, that is, echo ultrasonic waves.

The ultrasonic receiver 102 may include a microphone for detecting echo ultrasonic waves, and when the microphone is exposed to sound waves, a vibrating plate, a diaphragm (diaphragm microphone) or another sensing element of the microphone may react thereto. That is, the vibrating plate or diaphragm of the microphone may detect a change in air pressure caused by sound waves and convert the change into an electrical signal.

However, the ultrasonic receiver 102 may further include another component suitable for detecting and receiving echo ultrasonic waves, in addition to the microphone. For example, the ultrasonic receiver 102 may include a signal amplifier and/or a signal processor for amplifying a converted electrical signal.

The cleaning device 100 may include at least one ultrasonic receiver 102 and the at least one ultrasonic receiver 102 may be positioned between a plurality of ultrasonic generators implemented in a phased array form.

In this case, because a speed of echo ultrasonic waves depends on a temperature around a target, information on a temperature around contamination (or a target area of clothes accommodated inside the chamber 11) may be obtained based on information on a speed of echo ultrasonic waves received through the ultrasonic receiver 102. This will be described in detail with reference to FIG. 9, below.

The fan 103 may supply wind toward contamination. That is, the fan 103 may perform a cooling operation to cool heat generated by ultrasonic waves irradiated onto contamination (or a target area) by supplying wind toward the contamination.

More specifically, when ultrasonic waves irradiated from the ultrasonic irradiator 101 reaches contamination to perform a cleaning operation, fabric at a target area at which the contamination is located may absorb vibration energy of the ultrasonic waves as heat energy. Accordingly, heat damage to clothes may occur.

To prevent this, the fan 103 may supply wind, airflow, during or after the cleaning operation to cool the heat of the fabric and maintain a constant temperature range.

The cleaning device 100 may include at least one fan 103. A position and arrangement of the at least one fan 103 are not limited and the at least one fan 103 may be located at any position and arrangement that are suitable for performing a cooling operation.

The cleaning device 100 may further include a fan motor connected to the fan 103 to generate a rotation force of the fan 103.

The cleaning device 100 may include a plurality of moving portions 105 connected to the focusing device 200. The plurality of moving portions 105 may be spaced apart from each other along the first direction or the second direction. Referring to FIG. 4, two moving portions 105 may be arranged on left and right sides of the cleaning device 100 in such a way as to be spaced apart from each other in the first direction. However, this may be only an example, and other arrangements and numbers of the moving portions 105 may be possible as long as the cleaning device 100 is easily movable to focus on contamination while moving along the focusing device 200.

Also, the cleaning device 100 according to another embodiment may further include an air spray unit 104 that supplies outside air into the chamber 11 for a more powerful cooling operation.

The air spray unit 104 may receive outside air and supply relatively cooled air to contamination (or a target area) during a cooling operation.

The air spray unit 104 may include an air supply source, at least one air spray nozzle, and an airflow path connected to the air supply source and guiding supplied air to the air spray nozzle. In this case, the air supply source may be configured with an air pump, etc.

The air spray nozzle of the air spray unit 104 may be positioned adjacent to the fan 103. Therefore, cooled air supplied from the air spray unit 104 may be transferred to contamination by riding wind formed by the fan 103 without being spread to surroundings by convection.

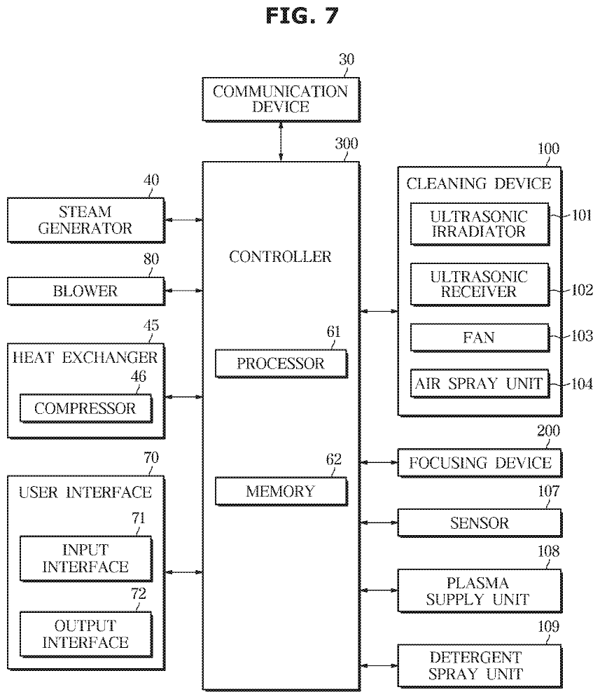

FIG. 7 is a control block diagram according to an embodiment.

Referring to FIG. 7, the clothes care apparatus 1 according to an embodiment may include a communication device 30, the steam generator 40, a blower 80, the heat exchanger 45, a user interface 70, the cleaning device 100, the sensor 107, the focusing device 200, the plasma supply unit 108, the detergent spray unit 109, and/or a controller 300.

The communication device 30 may include at least one of a short-range communication module or a long-distance communication module.

The communication device 30 may transmit data to an external device or receive data from the external device. For example, the communication device 30 may establish communication with a server, a user device and/or another home appliance and transmit/receive various data to/from the server, the user device and/or the other home appliance.

To this end, the communication device 30 may establish a direct (for example, wired) communication channel or a wireless communication channel with the external device, and support communication through the established communication channel. According to an embodiment, the communication device 30 may include a wireless communication module (for example, a cellular communication module, a short-range communication module, or a global navigation satellite system (GNSS) communication module) or a wired communication module (for example., a local area network (LAN) communication module or a power line communication module). A corresponding communication module among the communication modules may communicate with an external device through a first network (for example, a short-range communication network, such as Bluetooth, WiFi Direct, or Infrared data association (IrDA)) or a second network (for example, a long-distance communication network, such as a legacy cellular network, a 5-Generation (5G) network, a next-generation communication network, the Internet, or a computer network (for example, a LAN or a wide area network (WAN)). The various kinds of communication modules may be integrated into a single component (for example, a single chip) or implemented with a plurality of components (for example, a plurality of chips).

The short-range wireless communication module may include a Bluetooth communication module, a Bluetooth Low Energy (BLE) communication module, a Near Field Communication (NFC) communication module, a Wireless Local Area Network (WLAN; WiFi) communication module, a Zigbee communication module, an IrDA communication module, a Wi-Fi Direct (WFD) communication module, a Ultrawideband (UWB) communication module, an Ant+ communication module, a microwave (uWave) communication module, etc., although not limited thereto.

The long-distance wireless communication module may include a communication module that performs various kinds of long-distance communications, and may include a mobile communication device. The mobile communication device may transmit/receive a wireless signal to/from at least one of a base station, an external terminal, or a server on a mobile communication network.

According to an embodiment, the communication device 30 may communicate with an external device, such as a server, a user device, another home appliance, etc., through a surrounding Access Point (AP). The AP may connect a LAN to which the clothes care apparatus 1, another home appliance, and/or a user device is connected to a WAN to which a server is connected. The clothes care apparatus 1, the other home appliance, and/or the user device may be connected to the server through the WAN.

The steam generator 40 may spray steam into the chamber 11. The steam generator 40 may operate based on a control signal from the controller 300.

The blower 80 may include the first circulator 42 and/or the second circulator 75.

The first circulator 42 may discharge air inside the chamber 11 back into the chamber 11 via the machine room 13, the evaporator 46, and the condenser 47. The first circulator 42 may operate based on a control signal from the controller 300.

The second circulator 75 may discharge air inside the chamber 11 back into the chamber 11 via the clothes support member 50. The second circulator 75 may operate based on a control signal from the controller 300.

The heat exchanger 45 may be used to heat air supplied into the chamber 11 and may include the compressor 48 for compressing a refrigerant. The compressor 48 may operate based on a control signal from the controller 300.

The user interface 70 may include at least one input interface 70a and at least one output interface 70b.

The at least one input interface 70a may include, for example, a tact switch, a push switch, a slide switch, a toggle switch, a micro switch, a touch switch, a touch pad, a touch screen, a jog dial, and/or a microphone.

In the present disclosure, a ‘button’ may be replaced with a User Interface (UI) Element, a tact switch, a push switch, a slide switch, a toggle switch, a micro switch, a touch switch, a touch pad, a touch screen, a jog dial, and/or a microphone.

At least one output interface 70b may generate sensory information and transfer various information related to operations of the clothes care apparatus 1 to a user.

For example, the at least one output interface 70b may transfer information related to a clothes care course, an operation time of the clothes care apparatus 1, a target area setting of spot care, etc. to the user. Information related to operations of the clothes care apparatus 1 may be output through a display, an indicator, a voice, etc. The at least one output interface 70b may include, for example, a Liquid Crystal Display (LCD) panel, an indicator, a Light Emitting Diode (LED) panel, a speaker, etc.

The cleaning device 100 may be provided on one side wall of the chamber 11 to spot-clean clothes accommodated in the chamber 11.

The cleaning device 100 may include components (for example, the ultrasonic irradiator 101, the ultrasonic receiver 102, the fan 103, and/or the air spray unit 104) of the cleaning device 100. Each of the components of the cleaning device 100 may operate based on a control signal from the controller 300.

The sensor 107 may obtain information on contamination of clothes accommodated in the chamber 11 and transfer the obtained information to the controller 300.

The information on the contamination may include information on an area (that is, target area) of the clothes at which the contamination is located. The controller 300 may determine a target area based on the received information on the contamination.

For example, the sensor 107 may include a camera and image data collected by the camera may be transferred as information on contamination to the controller 300.

The focusing device 200 may move the cleaning device 100 to the target area inside the chamber 11 and the focusing device 200 may operate based on a control signal from the controller 300.

More specifically, the controller 300 may determine a location of a target area based on information received from the sensor 107 and control the focusing device 200 for the cleaning device 100 to focus on the target area.

The plasma supply unit 108 may supply plasma into the chamber 11 to remove contamination by allowing activated plasma particles to interact with contaminants.

The detergent spray unit 109 may spray a detergent or cleaning liquid toward contamination detected by the sensor 107 to increase cleaning power.

The plasma supply unit 108 or the detergent spray unit 109 may operate based on a control signal from the controller 300.

The controller 300 may be electrically connected to the communication device 30, the steam generator 40, the blower 80, the heat exchanger 45, the user interface 70, the cleaning device 100, the sensor 107, the focusing device 200, the plasma supply unit 108 and/or the detergent spray unit 109.

The controller 300 controlling the communication device 30, the steam generator 40, the blower 80, the heat exchanger 45, the user interface 70, the cleaning device 100, the sensor 107, the focusing device 200, the plasma supply unit 108 and/or the detergent spray unit 109 may include the controller 300 transmitting a control command to the communication device 30, the steam generator 40, the blower 80, the heat exchanger 45, the user interface 70, the cleaning device 100, the sensor 107, the focusing device 200, the plasma supply unit 108 and/or the detergent spray unit 109.

In the present disclosure, controlling the cleaning device 100 may include controlling the components (e.g., the ultrasonic irradiator 101, the ultrasonic receiver 102, the fan 103, and/or the air spray unit 104) of the cleaning device 100.

According to an embodiment, the controller 300 may control various components of the clothes care apparatus 1 to perform a clothes care course or a spot care course according to a user input. For example, the controller 300 may control the heat exchanger 45 and the first circulator 42 to supply hot air into the chamber 11, control the steam generator 40 to supply steam into the chamber 11, control the second circulator 75 to circulate air in the chamber 11, or control the cleaning device 100 to spot-clean clothes accommodated in the chamber 11.

The controller 300 may process data collected from various components (e.g., the user interface 70, the communication device 30, the cleaning device 100, and/or the sensor 107) of the clothes care apparatus 1. The controller 300 may process a user input received through the user interface 70 and perform an operation corresponding to the user input.

The controller 300 may include hardware, such as a central processing unit (CPU), Micom, a memory, etc., and software such as a control program. For example, the controller 300 may include at least one memory 62 that stores data in the form of an algorithm or program for controlling operations of components in the clothes care apparatus 1, and at least one processor 61 that performs the above-described operations and operations which will be described below by using the data stored in the at least one memory 62. The memory 62 and the processor 61 may be implemented with separate chips. The processor 310 may include one, two, or more processor chips or one, two, or more processing cores. The memory 62 may include one, two, or more memory chips or one, two, or more memory blocks. Also, the memory 62 and the processor 61 may be implemented with a single chip.

The at least one memory 62 may store algorithms for a clothes care course, a spot care course, and/or a course regarding a cleaning operation.

The controller 300 may be mounted on a printed circuit board provided on a rear surface of the user interface 70, although a location of the controller 300 is not limited thereto.

The controller 300 according to an embodiment may control, based on information on a target area, the at least one focusing device 200 to move the at least one cleaning device 100 to a location at which the at least one cleaning device 100 is capable of irradiating focused ultrasonic waves onto the target area.

The controller 300 may control the fan 103 based on a speed of echo ultrasonic waves received by the ultrasonic receiver 102.

The controller 300 may turn on the fan 103 based on the speed of the echo ultrasonic waves received by the ultrasonic receiver 102 being equal to or greater than a first reference value.

The controller 300 may determine a distance from each of the at least one cleaning device 100 to contamination based on a time length from a time at which the ultrasonic irradiator 101 has irradiated ultrasonic waves until a time at which the ultrasonic receiver 102 has received echo ultrasonic waves, and turn on the fan 103 included in at least one cleaning device 100 within a reference distance from the contamination among the at least one cleaning device 100.

The controller 300 may determine a control parameter of the fan 103 based on the speed of the echo ultrasonic waves received by the ultrasonic receiver 102.

The control parameter of the fan 103 may include at least one of revolutions per minute (RPM) of the fan motor, a rotation speed of a wind direction controller 113 or wind direction controller 123, or a blowing time.

The controller 300 may turn off the fan 103 based on the speed of the echo ultrasonic waves received by the ultrasonic receiver 102 being less than the first reference value.

The controller 300 may control the air spray unit 104 based on the speed of the echo ultrasonic waves received by the ultrasonic receiver 102.

Based on the speed of the echo ultrasonic waves received by the ultrasonic receiver 102 being equal to or less than a second reference value that is greater than the first reference value, the controller 300 may turn off the air spray unit 104 and turn on the fan 103.

Based on the speed of the echo ultrasonic waves received by the ultrasonic receiver 102 being greater than the second reference value, the controller 300 may turn on both the air spray unit 104 and the fan 103.

Based on the speed of the echo ultrasonic waves received by the ultrasonic receiver 102 being greater than the second reference value, the controller 300 may determine a control parameter of the air spray unit 104 according to the speed.

The control parameter of the air spray unit 104 may include at least one of a spray amount or a spray time.

Based on the speed of the echo ultrasonic waves received by the ultrasonic receiver 102 being less than the second reference value, the controller 300 may turn off the air spray unit 104.

The components shown in FIG. 7 may be an example of components of the clothes care apparatus 1 according to an embodiment, and the clothes care apparatus 1 according to an embodiment may further include another component in addition to the components shown in FIG. 7 or may omit some of the components shown in FIG. 7

For example, the clothes care apparatus 1 according to an embodiment may include an actuator for automatically opening the door 20, a door opening/closing sensor for detecting opening/closing of the door 20, a proximity sensor for detecting approach of an object to a certain location around the clothes care apparatus 1, etc.

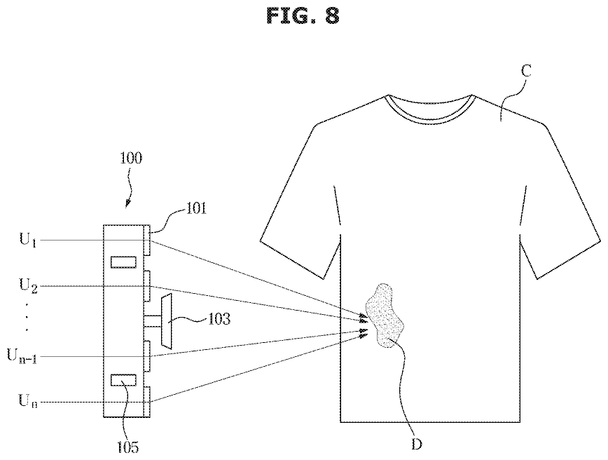

FIG. 8 is a diagram for describing how a cleaning device according to an embodiment irradiates ultrasonic waves onto contamination.

According to an embodiment, the controller 300 may, based on information received from the sensor 107, determine whether contamination D exists in clothes accommodated in the chamber 11 and an area (that is, a target area) at which the contamination D is located. Based on this, the controller 300 may control the focusing device 200 to move the cleaning device 100 to a location at which the cleaning device 100 is capable of irradiating focused ultrasonic waves onto the target area.

After the cleaning device 100 is positioned toward the target area, the cleaning device 100 may irradiate focused ultrasonic waves toward the contamination D based on a control signal from the controller 300.

Irradiating focused ultrasonic waves in the cleaning device 100 may include generating ultrasonic waves in the ultrasonic generator and adjusting a direction and form of an ultrasonic beam to a desired direction and form such that the generated ultrasonic waves are focused on the target area.

That is, the ultrasonic irradiator 101 may further include a beam former for adjusting a direction or pattern of ultrasonic waves to a specific direction or pattern.

More specifically, referring to FIG. 8, the beam former may adjust an irradiation time and phase of ultrasonic waves U1, U2, . . . , Un-1, Un generated in the ultrasonic generator. More specifically, the beam former may adjust a time and/or phase of ultrasonic waves irradiated from each ultrasonic generator based on a control signal from the controller 300 such that the ultrasonic waves interfere with each other or are strengthened and focused on the target area.

Accordingly, the cleaning device 100 may apply a mechanical force by concentrating ultrasonic energy on the target area while removing contamination using plasma, thereby improving cleaning power.

However, when the ultrasonic irradiator 101 removes the contamination by irradiating focused ultrasonic waves onto the target area, ultrasonic energy which has reached clothes C may be converted into heat energy due to friction and vibrations, and thus, heat damage to fabric may occur.

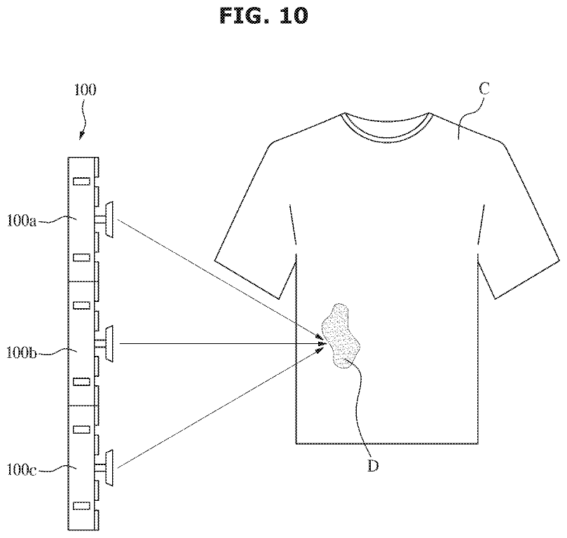

To prevent this, detecting heat generated when the ultrasonic irradiator 101 irradiates ultrasonic waves onto the clothes C and controlling the fan 103 or the air spray unit 104 based on the detected heat to perform a cooling operation for cooling the heat will be described with reference to FIG. 9 and FIG. 10, below.

FIG. 9 is a diagram for describing how a cleaning device according to an embodiment receives echo ultrasonic waves from contamination and detects heat of a contaminated area of clothes.

According to an embodiment, the ultrasonic receiver 102 may receive ultrasonic waves R1 and R2 reflected from contamination D among ultrasonic waves U1, U2, . . . , Un-1, Un irradiated toward the contamination D.

The ultrasonic waves reflected from the contamination D may be referred to as echo ultrasonic waves.

The controller 300 may detect a temperature of air around the contamination based on a speed of the echo ultrasonic waves received by the ultrasonic receiver 102. Because a propagation speed of ultrasonic waves changes depending on properties of a medium, a speed of ultrasonic waves being propagated through air or another medium changes according to a temperature. That is, as the temperature increases, the propagation speed may also increase, and as the temperature decreases, the propagation speed may also decrease.

More specifically, the controller 300 may determine a distance from the cleaning device 100 and the contamination D based on a time length from a time at which ultrasonic waves have been initially irradiated until a time at which echo ultrasonic waves have been received. At this time, the ultrasonic waves initially irradiated from the ultrasonic irradiator 101 may have a preset speed.

Thereafter, the controller 300 may repeatedly irradiate ultrasonic waves to measure, for each ultrasonic-wave irradiation event, a time length from a time at which the ultrasonic irradiator 101 has irradiated ultrasonic waves until a time at which the ultrasonic receiver 102 has received echo ultrasonic waves.

Accordingly, the controller 300 may measure a speed of the echo ultrasonic waves based on the determined distance and the time length from the time at which the ultrasonic waves have been irradiated until the time at which the echo ultrasonic waves have been received.

As described above, because a speed of echo ultrasonic waves changes according to a temperature, the controller 300 may detect heat generation by measuring the speed of the echo ultrasonic waves.

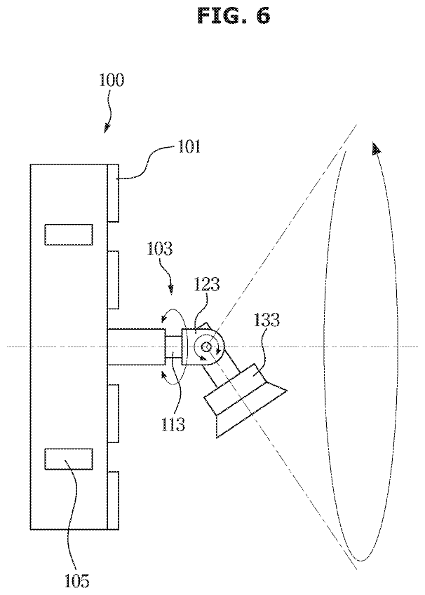

FIG. 6 is a diagram for describing a fan or an air spray unit included in a cleaning device according to an embodiment.

According to an embodiment, based on a measured speed of echo ultrasonic waves received by the ultrasonic receiver 102 being equal to or greater than a preset reference value, the controller 300 may control the fan 103 or the air spray unit 104 to perform a cooling operation.

That is, based on a measured speed of echo ultrasonic waves being equal to or greater than the preset reference value, the controller 300 may determine that heat that may damage clothes has been generated in a target area and control the cleaning device 100 to perform a cooling operation for cooling the heat.

Accordingly, the cleaning device 100 may increase cleaning power by irradiating ultrasonic waves to apply an indirect mechanical force and simultaneously prevent damage to fabric by preventing a temperature of the target area from exceeding a preset temperature.

The cleaning device 100 according to an embodiment may control the fan 103 to perform a cooling operation.

Referring to FIG. 6, the fan 103 may include a first wind direction controller 113 and a second wind direction controller 123 for controlling a blowing direction of the fan 103, and/or a head 133 connected to one ends of the wind direction controller 113 and wind direction controller 123 and generating wind for blowing. In the present disclosure, the wind direction controller 113 and wind direction controller 123 are shown as including the first wind direction controller 113 that rotates in an up-down direction and the second wind direction controller 123 that rotates in a left-right direction. However, the wind direction controller 113 and wind direction controller 123 are not limited thereto and may include any one of the first wind direction controller 113 or the second wind direction controller 123.

Also, the fan motor may be provided inside the cleaning device 100 or on a rear side of the cleaning device 100, which is not shown.

The wind direction controller 113 and wind direction controller 123 may be coupled to the fan motor and rotate by receiving a driving force from the fan motor according to a control signal from the controller 300. Accordingly, the wind direction controller 113 and wind direction controller 123 may increase efficiency of a cooling operation by adjusting a direction of the head 133 more precisely toward contamination.

Also, the wind direction controller 113 and wind direction controller 123 may repeatedly rotate to repeatedly rotate the head 133 and thereby form a wide range of airflow. At this time, as a rotation speed of the wind direction controller 113 and wind direction controller 123 increases, a stronger airflow may be formed.

That is, the controller 300 controlling the fan 103 may include controlling the fan motor of the fan 103 or the wind direction controller 113 and wind direction controller 123 of the fan 103 to adjust RPM of the fan 103, a blowing time, or a rotation speed of the wind direction controller 113 and wind direction controller 123.

According to an embodiment, the cleaning device 100 may further the air spray unit 104 that supplies outside air to a contaminated area to increase efficiency of a cooling operation.

The air spray unit 104 may include at least one air spray nozzle, and the air spray nozzle may be positioned adjacent to the fan 103 to, when the fan 103 is turned on and thus a cooling operation is performed, supply cooled air to contamination. The air spray nozzle may have various materials or shapes.

The air spray unit 104 may include a spray valve to adjust an air spray amount or an air spray time. The spray valve may be connected to the air spray nozzle and adjusted according to a control signal from the controller 300.

That is, the controller 300 controlling the air spray unit 104 may include controlling the spray valve to adjust an air spray amount or an air spray time.

FIG. 10 shows an example in which a clothes care apparatus according to an embodiment includes a plurality of cleaning devices.

According to an embodiment, the clothes care apparatus 1 may include at least one cleaning device 100.

In a case where the clothes care apparatus 1 includes a plurality of cleaning devices 100, the cleaning devices 100 may be implemented in an array form with at least one row and column.

In the case where the plurality of cleaning devices 100 are implemented in the array form, the controller 300 may determine a distance between each cleaning device 100 and contamination D based on a speed of echo ultrasonic waves received by the ultrasonic receiver 102.

The controller 300 may determine at least one cleaning device 100 within a reference distance range from the contamination D among the at least one cleaning device 100, turn on the fan 103 of the at least one cleaning device 100 within the reference distance range, and turn off the fan 103 of the remaining cleaning device 100 outside the distance range.

Here, the reference distance may be a value set in advance according to fabrics of clothes and stored in the memory 62 when the clothes care apparatus 1 has been manufactured, or may be a value set in advance by a user through the user interface 70. Also, the reference distance may change or be updated through communication with a server or another user device via the communication device 30 or by a machine learning model.

For example, as shown in FIG. 10, the plurality of cleaning devices 100 may form three rows and one column and irradiate focused ultrasonic waves onto a target area.

In a case where the reference distance is d′, a distance from cleaning devices 100a and 100c positioned in first and third columns to contamination D is d1, and a distance from a cleaning device 100b positioned in a second column to the contamination D is d2, and d2<d′<d1, the controller 300 may perform control of turning on the fan 103 included in the cleaning device 100b positioned in the second column among the plurality of cleaning devices 100a, 100b, and 100c.

That is, by turning on the fan 103 positioned adjacent to the contamination D and capable of effectively performing a cooling operation, energy efficiency may increase.

However, in the case where the clothes care apparatus 1 includes the plurality of cleaning devices 100, the plurality of cleaning devices 100 may be arranged in other various forms inside the chamber 11, instead of the above-described form. For example, some of the plurality of cleaning devices 100 may be positioned on one side of the chamber 11 and the remaining cleaning devices 100 may be positioned on the other sides of the chamber 11. Each of three devices positioned in the respective sides may include the focusing device 200. However, it is obvious to a person skilled in the art that the plurality of cleaning devices 100 are embodied in any form and location suitable for removing contamination D.

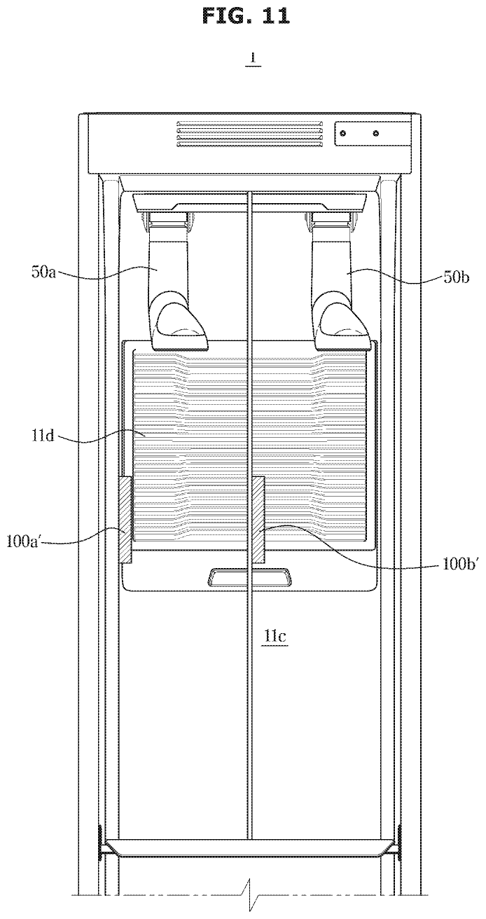

FIG. 11 shows an example in which a clothes care apparatus according to an embodiment includes a plurality of chambers and at least one cleaning device included in each chamber.

The clothes care apparatus 1 according to an embodiment may include a plurality of chambers 11 partitioned by a partition wall. Each chamber 11 may include at least one cleaning device 100 that removes contamination of clothes accommodated in the chamber 11. Accordingly, each chamber 11 may include, to independently remove contamination and perform a cooling operation, the least one sensor 107 that obtains information on contamination of clothes accommodated in the chamber 11 and the focusing device 200 that causes the corresponding cleaning device 100 to focus on a target area. In addition, each chamber 11 may further include a component required to remove contamination and perform a cooling operation or may omit some of the above-described components. However, components included in the plurality of chambers 11 may not be necessarily the same.

The controller 300 may independently control cleaning device 100a′ and cleaning device 100b′ and other components included in respective ones of chamber 11a′ and chamber 11b′. In a case where clothes accommodated in the respective ones of chamber 11a′ and chamber 11b′ include contamination at different areas (that is, target areas), the controller 300 may move the respective cleaning device 100a′ and cleaning device 100b′ to cause the respective cleaning device 100a′ and cleaning device 100b′ to focus on the respective target areas.

Also, the controller 300 may independently control on/off of fan 103a′ and fan 103b′ or air spray unit 109a′ and air spray unit 109b′ included in the respective cleaning device 100a′ and cleaning device 100b′ based on speeds of echo ultrasonic waves received by ultrasonic receiver 102a′ and ultrasonic receiver 102b′ of the cleaning device 100a′ and cleaning device 100b′. In addition, the controller 300 may determine different control parameters for the fan 103a′ and fan 103b′ or the air spray unit 109a′ and air spray unit 109b′ included in the respective cleaning device 100a′ and cleaning device 100b′.

Accordingly, the clothes care apparatus 1 having the plurality of chambers 11 may remove contamination of a plurality of pieces of clothes at once, thereby increasing clothes care efficiency.

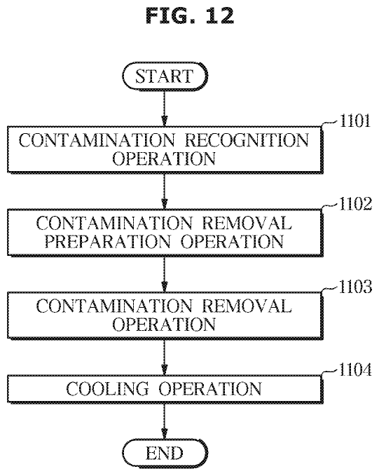

FIG. 12 is a flowchart illustrating a cleaning operation according to an embodiment.

According to an embodiment, the controller 300 may receive information on contamination obtained by the sensor 107 and determine whether contamination D exists in clothes C accommodated in the chamber 11 and a location (that is, a target area) at which the contamination D exists (operation 1101).

To this end, the memory 62 may store an algorithm for recognizing contamination D based on information obtained by the sensor 107 and determining a target area. For example, the controller 300 may input information obtained by the sensor 107 to a machine learning model to determine whether contamination D exists and identify a target area.

For example, the controller 300 may determine whether contamination D exists and identify a target area based on image data in a case where the sensor 107 includes a camera or based on light quantity data received by the sensor 107 in a case where the sensor 107 includes an optical sensor.

After the controller 300 determines that contamination D exists and identifies a target area, the controller 300 may control the focusing device 200 to perform a preparation operation for removing the contamination (operation 1102).

For example, the controller 300 may control the focusing device 200 to move the cleaning device 100 to a position at which the cleaning device 100 is capable of irradiating focused ultrasonic waves toward the contamination D.

At this time, in a case where the clothes care apparatus 1 includes a plurality of cleaning devices 100 or a plurality of focusing devices 200, the controller 300 may independently control the plurality of focusing devices 200 to cause the respective cleaning devices 100 to focus on respective target areas.

After the controller 300 determines that the cleaning device 100 has been located at the position at which the cleaning device 100 is capable of irradiating focused ultrasonic waves toward the contamination D or the target area (that is, after the controller 300 determines that a preparation operation for removing contamination has terminated), the controller 300 may perform an operation for removing contamination (operation 1103).

The controller 300 may control the plasma supply unit 108 to supply plasma for removing the contamination into the chamber 11.

Also, the controller 300 may control the cleaning device 100 to generate ultrasonic waves through the ultrasonic irradiator 101 and irradiate the ultrasonic waves toward the contamination D. At this time, as described above, the controller 300 may set a focal point to correspond to the detected contamination and control the beam former to adjust a time and phase of the ultrasonic waves in order to irradiate focused ultrasonic waves onto the contamination.

Before the ultrasonic irradiator 101 irradiates ultrasonic waves or while the ultrasonic irradiator 101 irradiates ultrasonic waves, the controller 300 may control the detergent spray unit 109 to spray a cleaning liquid toward the contamination D. The controller 300 may improve cleaning power by spraying the cleaning liquid toward the contamination D.

The controller 300 may perform a cooling operation to cool heat generated in the target area when energy transferred in the form of ultrasonic waves is converted into heat energy (operation 1104).

At this time, the cooling operation may be performed after or during a contamination removal operation.

The controller 300 may control the fan 103 or the air spray unit 104 of the cleaning device 100 to perform the cooling operation.

More specifically, the controller 300 may control on/off of the fan 103 or the air spray unit 104 based on a speed of echo ultrasonic waves received by the ultrasonic receiver 102 to prevent a temperature of the target area from rising above a preset upper limit temperature. In addition, the controller 300 may control the fan motor or the wind direction controller 113 and wind direction controller 123 of the fan 103 to adjust RPM of the fan 103, a blowing time, or rotation speeds of the wind direction controller 113 and wind direction controller 123. In addition, the controller 300 may control the air spray unit 104 to adjust an air spray amount or a spray time.

Accordingly, the clothes care apparatus 1 may irradiate ultrasonic waves while removing contamination using plasma, thereby increasing contamination removal efficiency and preventing damage to clothes.

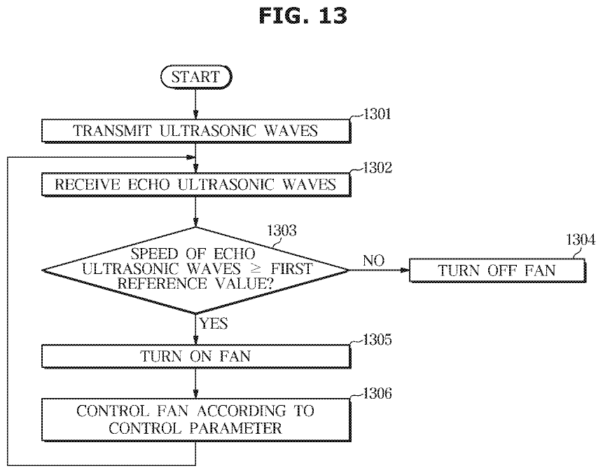

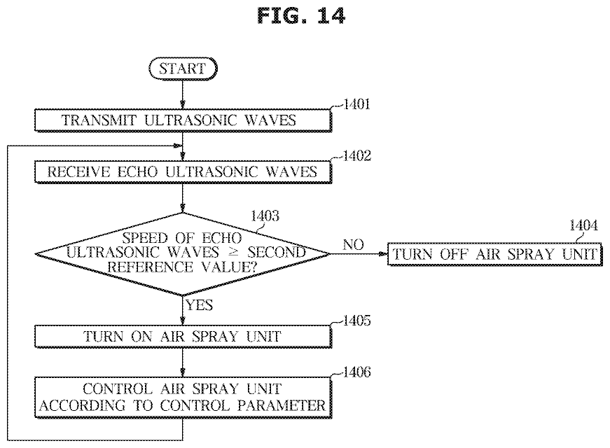

Hereinafter, the controller 300 controlling the fan 103 or the air spray unit 104 in a cooling operation will be described in detail with reference to FIG. 13 and FIG. 14.

FIG. 13 is a flowchart illustrating controlling a fan included in a cleaning device in the cooling operation of FIG. 12 according to an embodiment.

According to an embodiment, the cleaning device 100 may irradiate focused ultrasonic waves toward contamination D based on a control signal from the controller 300. That is, the controller 300 may control the ultrasonic irradiator 101 to transmit ultrasonic waves toward the contamination D (operation 1301).

The controller 300 may control the ultrasonic receiver 102 to receive ultrasonic waves (that is, echo ultrasonic waves) reflected from the contamination D among ultrasonic waves transmitted toward the contamination D (operation 1302).

As described above with reference to FIG. 8, the controller 300 may measure a time length from a time at which the ultrasonic irradiator 101 has irradiated ultrasonic waves until a time at which the ultrasonic receiver 102 has received echo ultrasonic waves. Thereafter, the controller 300 may measure a speed of the echo ultrasonic waves based on the measured time length and a distance between the cleaning device 100 and the contamination D, the distance being determined when the ultrasonic waves have been initially irradiated.

The controller 300 may determine whether the measured speed of the echo ultrasonic waves is equal to or greater than a first reference value (operation 1303). The first reference value may be a value set in advance and stored in the memory 62 when the clothes care apparatus 1 has been manufactured, or may be a value set in advance by a user through the user interface 70. Also, the first reference value may change or be updated through communication with a server or another user device via the communication device 30 or by a machine learning model.

According to the speed of the echo ultrasonic waves being less than the first reference value (NO in 1303), the controller 300 may turn off the fan 103 (operation 1304). That is, according to the speed of the echo ultrasonic waves being less than first reference value, the controller 300 may determine that heat sufficient to damage a target area of clothes has not yet been generated and may not operate the fan 103.

In contrast, according to the speed of the echo ultrasonic waves being equal to or greater than the first reference value (YES in 1303), the controller 300 may turn on the fan 103 (operation 1305). The controller 300 turning on the fan 103 may include controlling driving of the fan motor to generate a rotation force of the fan 103.

When the fan 103 is turned on, the controller 300 may determine a control parameter of the fan 103 based on the calculated speed of the echo ultrasonic waves. The control parameter of the fan 103 may include at least one of RPM of the fan motor, a driving time, or rotation speeds of the wind direction controller 113 and wind direction controller 123.

For example, the controller 300 may control RPM (that is, a rotation speed) of the fan motor according to a difference between the measured speed of the echo ultrasonic waves and the first reference value.

That is, the controller 300 may control RPM of the fan motor to increase as the difference between the measured speed of echo ultrasonic waves and the first reference value increases. Also, when the fan 103 is turned on and wind is supplied to the target area by the fan 103, a measured speed of echo ultrasonic waves may decrease as air around the contamination becomes cold. Accordingly, the controller 300 may decrease RPM of the fan motor as a difference between a measured speed of echo ultrasonic waves and the first reference value decreases.

Also, the controller 300 may control an operating time of the fan motor according to a difference between a measured speed of echo ultrasonic waves and the first reference value. That is, as a difference between a measured speed of echo ultrasonic waves and the first reference value becomes greater, the controller 300 may determine that a cooling operation for cooling heat needs to be performed for a relatively longer time. Accordingly, the controller 300 may set an operating time of the fan motor based on a measured speed of echo ultrasonic waves.

Also, the controller 300 may control rotation speeds of the wind direction controller 113 and wind direction controller 123 among control parameters of the fan 103 based on a measured speed of echo ultrasonic waves.

The fan 103 according to an embodiment may include the first wind direction controller 113 and the second wind direction controller 123 for controlling a blowing direction of the fan 103, and/or the head 133 connected to one ends of the wind direction controller 113 and wind direction controller 123 and generating wind for blowing. In the present disclosure, the wind direction controller 113 and wind direction controller 123 are shown as including the first wind direction controller 113 that rotates in the up-down direction and the second wind direction controller 123 that rotates in the left-right direction. However, the wind direction controller 113 and wind direction controller 123 are not limited thereto and may include any one of the first wind direction controller 113 or the second wind direction controller 123.

The wind direction controller 113 and wind direction controller 123 may be coupled to the fan motor and rotate by receiving a driving force from the fan motor according to a control signal from the controller 300. Also, the head 133 may rotate according to a rotation of the wind direction controller 113 and wind direction controller 123 to form a wide range of airflow.

The controller 300 may control rotation speeds of the wind direction controller 113 and wind direction controller 123 according to a difference between a measured speed of echo ultrasonic waves and the first reference value. For example, as a difference between a measured speed of echo ultrasonic waves and the first reference value becomes greater, the controller 300 may increase rotation speeds of the wind direction controller 113 and wind direction controller 123. Accordingly, the fan 103 may form a stronger airflow to perform a strong cooling operation.

In addition, when a decrease in measured speed of echo ultrasonic waves is measured as air around contamination becomes colder, the controller 300 may decrease rotation speeds of the wind direction controller 113 and wind direction controller 123 as a difference between a measured speed of echo ultrasonic waves and the first reference value decreases.

A control parameter determined by the controller 300 after the fan 103 is turned on is not limited to the above-mentioned examples and any parameter that may affect operations of the fan 103 during a cooling operation may be controlled by the controller 300 based on a speed of echo ultrasonic waves received by the ultrasonic receiver 102.

The controller 300 may control the fan 103 according to the determined control parameter of the fan 103 (operation 1306).

The controller 300 may control the fan 103 to perform a cooling operation while continuing to monitor a speed of echo ultrasonic waves received by the ultrasonic receiver 102.

At this time, according to a measured speed of echo ultrasonic waves decreasing below the first reference value, the controller 300 may turn off the fan 103 to terminate the cooling operation.

That is, the controller 300 may control the fan 103 such that a speed of echo ultrasonic waves reflected from the contamination D is less than the first reference value and maintain a temperature around the contamination D or a temperature of a target area at which the contamination D is located below a preset temperature.

FIG. 14 is a flowchart illustrating controlling an air spray unit included in the cleaning device in the cooling operation of FIG. 12 according to an embodiment.

The cleaning device 100 according to an embodiment may further include the air spray unit 104 that supplies outside air into the chamber 11.

The air spray unit 104 may supply outside air into the chamber 11 by a control signal from the controller 300 to increase efficiency of a cooling operation. Here, the outside air may include cooled air compared to air inside the chamber 11.

The controller 300 controlling the air spray unit 104 based on a speed of echo ultrasonic waves received through the ultrasonic receiver 102 may include adjusting an air spray amount or an air spray time by controlling the spray valve.

Operation 1301 and operation 1302 of FIG. 13 described above may correspond to operation 1401 and operation 1402 of FIG. 14.

That is, the controller 300 may transmit ultrasonic waves toward the contamination (operation 1401), receive echo ultrasonic waves reflected among the ultrasonic waves (operation 1402), and measure a speed of the echo ultrasonic waves, which has been described with reference to FIG. 13.

The controller 300 may determine whether the measured speed of the echo ultrasonic waves is equal to or greater than a second reference value (operation 1403). The second reference value may be a value set in advance and stored in the memory 62 when the clothes care apparatus 1 has been manufactured, or may be a value set in advance by a user through the user interface 70. Also, the second reference value may change or be updated through communication with a server or another user device via the communication device 30 or by a machine learning model.

According to the speed of the echo ultrasonic waves received by the ultrasonic receiver 102 being equal to or greater than the first reference value and less than the second reference value (NO in operation 1403), the controller 300 may turn off the air spray unit 104 (operation 1404).

Here, the second reference value may be greater than the first reference value.

Accordingly, based on the speed of the echo ultrasonic waves received by the ultrasonic receiver 102 being equal to or greater than the first reference value and less than the second reference value (NO in operation 1403), the controller 300 may turn on only the fan 103 and turn off the air spray unit 104.

According to the speed of the echo ultrasonic waves received by the ultrasonic receiver 102 being less than the first reference value (NO in operation 1403), the controller 300 may turn off both the fan 103 and the air spray unit 104.

In contrast, according to the speed of the echo ultrasonic waves received by the ultrasonic receiver 102 being equal to or greater than the second reference value (YES in operation 1403), the controller 300may turn on the air spray unit 104 (operation 1405). That is, the controller 300 may turn on both the fan 103 and the air spray unit 104. In the case where the speed of the echo ultrasonic waves received by the ultrasonic receiver 102 is equal to or greater than the second reference value, an atmosphere around the contamination D may have been heated more than in the case where a speed of echo ultrasonic waves received by the ultrasonic receiver 102 is equal to or greater than the first reference value and less than the second reference value. That is, as there is a high risk that a target area of clothes C will be damaged by heat, the air spray unit 104 may be turned on together with the fan 103 to perform a more powerful cooling operation.

When the air spray unit 104 is turned on, the controller 300 may determine a control parameter of the air spray unit 104 based on a calculated speed of echo ultrasonic waves. The control parameter of the air spray unit 104 may include at least one of a spray amount or a spray time.

For example, the controller 300 may control a spray amount of air that is sprayed through the air spray unit 104 according to a difference between a measured speed of echo ultrasonic waves and the second reference value. At this time, the controller 300 controlling a spray amount of air that is sprayed through the air spray unit 104 may include the controller 300 adjusting a spray amount of air sprayed per unit time by controlling the spray valve of the air spray unit 104.

As the spray amount increases, more cooled air may be supplied into the chamber 11. Therefore, as a difference between a measured speed of echo ultrasonic waves and the second reference value becomes greater, the controller 300 may perform control of increasing a spray amount. Also, when the air spray unit 104 is turned on and wind is supplied to the target area by the air spray unit 104, air around the contamination may be cooled and thus, a measured speed of echo ultrasonic waves may decrease. Accordingly, as a difference between a measured speed of echo ultrasonic waves and the second reference value decreases, the controller 300 may reduce a spray amount.

Also, the controller 300 may control a spray time of the air spray unit 104 according to a difference between a measured speed of echo ultrasonic waves and the second reference value. That is, as a difference between a measured speed of echo ultrasonic waves and the second reference value becomes greater, the controller 300 may determine that a cooling operation for cooling heat needs to be performed for a relatively longer time. Accordingly, the controller 300 may set a spray time of the air spray unit 104 based on a measured speed of echo ultrasonic waves.

Control parameters of the air spray unit 104 determined by the controller 300 after the air spray unit 104 is turned on is not limited to the above-mentioned examples, and any parameter that may affect operations of the air spray unit 104 during a cooling operation may be controlled by the controller 300 based on a speed of echo ultrasonic waves received by the ultrasonic receiver 102.

The controller 300 may control the air spray unit 104 according to the determined control parameter of the air spray unit 104 (operation 1406).

The controller 300 may perform a cooling operation by controlling the air spray unit 104 while continuing to monitor a speed of echo ultrasonic waves received by the ultrasonic receiver 102.

At this time, when a measured speed of echo ultrasonic waves decreases below the second reference value, the controller 300 may turn off the fan 103. However, when a speed of echo ultrasonic waves is less than the second reference value and equal to or greater than the first reference value, the controller 300 may perform a cooling operation in a turned-on state of the fan 103.

That is, when a speed of echo ultrasonic waves reflected from the contamination D is equal to or greater than the second reference value, the controller 300 may control the air spray unit 104 together with the fan 103 in order to rapidly decrease a temperature of the target area through a strong cooling operation, thereby maintaining a temperature around the contamination D or a temperature of the target area at which the contamination is located below a preset temperature.

Meanwhile, the disclosed embodiments may be implemented in the form of a recording medium that stores instructions executable by a computer. The instructions may be stored in the form of program codes, and when executed by a processor, the instructions may create a program module to perform operations of the disclosed embodiments. The recording medium may be implemented as a computer-readable recording medium.

The computer-readable recording medium may include all kinds of recording media storing instructions that can be interpreted by a computer. For example, the computer-readable recording medium may be Read Only Memory (ROM), Random Access Memory (RAM), a magnetic tape, a magnetic disc, a flash memory, an optical data storage device, etc.

Also, the computer-readable recording medium may be provided in the form of a non-transitory storage medium, wherein the term ‘non-transitory storage medium’ simply means that the storage medium is a tangible device, and does not include a signal (e.g., an electromagnetic wave), but this term does not differentiate between where data is semi-permanently stored in the storage medium and where the data is temporarily stored in the storage medium. For example, the ‘non-transitory storage medium’ may include a buffer in which data is temporarily stored.

According to an embodiment, a method according to various embodiments disclosed in this document may be included and provided in a computer program product. The computer program product may be traded as a product between a seller and a buyer. The computer program product may be distributed in the form of a machine-readable recording medium (e.g., compact disc read only memory (CD-ROM)), or be distributed (e.g., downloadable or uploadable) online via an application store (e.g., Play Store™) or between two user devices (e.g., smart phones) directly. When distributed online, at least part of the computer program product (e.g., a downloadable app) may be temporarily generated or at least temporarily stored in the machine-readable recording medium, such as a memory of the manufacturer's server, a server of the application store, or a relay server.

A clothes care apparatus 1 according to an embodiment may include: a chamber 11 accommodating clothes; a plasma supply unit 108 configured to spray plasma into the chamber 11; at least one sensor 107 configured to obtain information on contamination of the clothes accommodated in the chamber 11; and at least one cleaning device 100 configured to remove the contamination, wherein the information on the contamination may include information on a target area at which the contamination is located on the clothes, and the cleaning device may include an ultrasonic irradiator 101 configured to irradiate ultrasonic waves onto the target area, an ultrasonic receiver 102 configured to receive echo ultrasonic waves reflected from the target area, a fan 103 configured to blow air to the target area, and a fan motor configured to generate a rotation force of the fan.

The clothes care apparatus 1 may further include at least one focusing device 200 configured to move each of the at least one cleaning device inside the chamber; and a controller 300 electrically connected to the plasma supply unit 108, the at least one sensor 107, the at least one cleaning device 100, and the at least one focusing device 200, and the controller 300 may be configured to, based on the information on the target area, control the at least one focusing device to move the at least one cleaning device to a location at which the at least one cleaning device is capable of irradiating focused ultrasonic waves onto the target area.

The controller 300 may be configured to turn on the fan 103 based on a speed of echo ultrasonic waves received by the ultrasonic receiver 102 being equal to or greater than a first reference value.

The controller 300 may configured to determine a distance from each of the at least one cleaning device 100 to the contamination based on a time length from a time at which the ultrasonic irradiator 101 has irradiated the ultrasonic waves until a time at which the ultrasonic receiver 102 has received the echo ultrasonic waves, and turn on the fan 103 included in at least one cleaning device 100 within a reference distance range from the contamination among the at least one cleaning device.

The controller 300 may determine a control parameter of the fan 103 based on the speed of the echo ultrasonic waves received by the ultrasonic receiver 102.

The control parameter of the fan 103 may include at least one of RPM of the fan motor, rotation speeds of wind direction controllers 113 and 123, or a blowing time.

The controller 300 may be configured to, based on the speed of the echo ultrasonic waves received by the ultrasonic receiver 102 being less than the first reference value, turn off the fan 103.

The controller 300 may control an air spray unit 104 based on the speed of the echo ultrasonic waves received by the ultrasonic receiver 102.

The controller 300 may be configured to, based on the speed of the echo ultrasonic waves received by the ultrasonic receiver 102 being less than a second reference value that is equal to or greater than the first reference value, turn off the air spray unit 104 and turn on the fan 103.

The controller 300 may be configured to, based on the speed of the echo ultrasonic waves received by the ultrasonic receiver 102 being equal to or greater than the second reference value, turn on both the air spray unit 104 and the fan 103.

The controller 300 may be configured to, based on the speed of the echo ultrasonic waves received by the ultrasonic receiver 102 being equal to or greater than the second reference value, determine a control parameter of the air spray unit 104 according to the speed.

The control parameter of the air spray unit 104 may include at least one of a spray amount or a spray time.

The controller 300 may be configured to, based on the speed of the echo ultrasonic waves received by the ultrasonic receiver 102 being less than the second reference value, turn off the air spray unit 104.

A control method of a clothes care apparatus 1 according to an embodiment may include: obtaining information on contamination of clothes accommodated in a chamber 11; irradiating ultrasonic waves onto the contamination in an ultrasonic irradiator 101; receiving echo ultrasonic waves reflected from the contamination by an ultrasonic receiver 102; and controlling a fan 103 included in a cleaning device 100 based on a speed of the echo ultrasonic waves received by the ultrasonic receiver 102.

The controlling of the fan 103 included in the cleaning device 100 based on the speed of the echo ultrasonic waves may include, based on the speed of the echo ultrasonic waves received by the ultrasonic receiver 102 being equal to or greater than a first reference value, turning on the fan 103.

The controlling of the fan 103 included in the cleaning device 100 based on the speed of the echo ultrasonic waves may include determining a control parameter of the fan 103 based on the speed of the echo ultrasonic waves received by the ultrasonic receiver 102, and the control parameter of the fan 103 may include at least one of RPM of the fan motor, a driving time, or rotation speeds of wind direction controller 113 and wind direction controller 123.

The controlling of the fan 103 included in the cleaning device 100 based on the speed of the echo ultrasonic waves may include, based on the speed of the echo ultrasonic waves received by the ultrasonic receiver 102 being less than a first reference value, turning off the fan.

The control method of the clothes care apparatus 1 may further include controlling an air spray unit 104 based on the speed of the echo ultrasonic waves received by the ultrasonic receiver 102.

The controlling of the fan 103 or the air spray unit 104 based on the speed of the echo ultrasonic waves may include, based on the speed of the echo ultrasonic waves received by the ultrasonic receiver 102 being less than a second reference value that is equal to or greater than the first reference value, turning off the air spray unit 104 and turning on the fan 103.

The controlling of the fan 103 or the air spray unit 104 based on the speed of the echo ultrasonic waves may include, based on the speed of the echo ultrasonic waves received by the ultrasonic receiver 102 being equal to or greater than the second reference value, turning on both the air spray unit 104 and the fan 103.

The clothes care apparatus 1 according to an aspect of the present disclosure may maintain a temperature of a target area of clothes at which contamination is located within a preset range to increase efficiency of removing the contamination from the clothes by applying an indirect physical force.

The clothes care apparatus 1 according to an aspect of the disclosure may minimize damage to clothes while indirectly applying a physical force to the clothes in a cleaning operation using plasma.

Technical aspects achieved by the present disclosure are not limited to those mentioned above, and other technical aspects not mentioned may be clearly understood by one of ordinary skill in the technical art to which the present disclosure belongs from the following description.

Although example embodiments have been described with reference to the accompanying drawings, it will be understood by one of ordinary skill in the technical art to which the present disclosure belongs that the present disclosure can be implemented in different forms from the described embodiments without changing the technical spirit or essential features of the present disclosure. Thus, it should be understood that the described embodiments are merely for illustrative purposes and not for limitation purposes.

What is claimed is:

1. A clothes care apparatus comprising:
a chamber configured to accommodate clothes;
a plasma supply unit configured to spray plasma into the chamber;
at least one sensor configured to obtain information regarding contamination of the clothes in a state that the clothes are accommodated in the chamber; and
at least one cleaning device configured to remove the contamination,
wherein the information related to the contamination comprises information related to a target area at which the contamination is located on the clothes, and
wherein the at least one cleaning device comprises:
an ultrasonic irradiator configured to irradiate ultrasonic waves onto the target area,
an ultrasonic receiver configured to receive echo ultrasonic waves reflected from the target area,
a fan configured to blow air to the target area, and
a fan motor configured to generate a rotation force of the fan.

2. The clothes care apparatus of claim 1, further comprising:
at least one focusing device configured to move the at least one cleaning device inside the chamber; and
a controller comprising a processor, the controller being operatively connected to the plasma supply unit, the at least one sensor, the at least one cleaning device, and the at least one focusing device,
wherein the controller is configured to, based on the information regarding the target area, control the at least one focusing device to move the at least one cleaning device to a location at which the at least one cleaning device is capable of irradiating the ultrasonic waves onto the target area.

3. The clothes care apparatus of claim 2, wherein the controller is further configured to, based on a speed of the echo ultrasonic waves received by the ultrasonic receiver being equal to or greater than a first reference value, turn on the fan.

4. The clothes care apparatus of claim 3, wherein the controller is further configured to:
determine, based on a time length from a first time at which the ultrasonic irradiator irradiates the ultrasonic waves until a second time at which the ultrasonic receiver receives the echo ultrasonic waves, a distance from the at least one cleaning device to the contamination, and
turn on the fan within a reference distance range from the contamination.

5. The clothes care apparatus of claim 3, wherein the at least one cleaning device further comprises a wind direction controller coupled to the fan motor and configured to adjust a blowing direction of the fan, and
wherein the controller is further configured to determine a control parameter of the fan according to the speed of the echo ultrasonic waves received by the ultrasonic receiver.

6. The clothes care apparatus of claim 5, wherein the control parameter of the fan comprises at least one of Revolutions Per Minute (RPM) of the fan motor, a driving time, or a rotation speed of the wind direction controller.

7. The clothes care apparatus of claim 6, wherein
the controller is further configured to, based on the speed of the echo ultrasonic waves received by the ultrasonic receiver being less than the first reference value, turn off the fan.

8. The clothes care apparatus of claim 2, further comprising:
an air spray unit configured to supply outside air into the chamber,
wherein the controller is further configured to control the air spray unit based on a speed of the echo ultrasonic waves received by the ultrasonic receiver.

9. The clothes care apparatus of claim 8, wherein
the controller is further configured to, based on the speed of the echo ultrasonic waves received by the ultrasonic receiver being less than a second reference value that is equal to or greater than a first reference value, turn off the air spray unit and turn on the fan.

10. The clothes care apparatus of claim 8, wherein the controller is further configured to, based on the speed of the echo ultrasonic waves received by the ultrasonic receiver being equal to or greater than a second reference value, turn on both the air spray unit and the fan.

11. The clothes care apparatus of claim 10, wherein the controller is further configured to, based on the speed of the echo ultrasonic waves received by the ultrasonic receiver being equal to or greater than the second reference value, determine a control parameter of the air spray unit.

12. The clothes care apparatus of claim 11, wherein the control parameter of the air spray unit comprises at least one of a spray amount or a spray time.

13. The clothes care apparatus of claim 12, wherein the controller is further configured to, based on the speed of the echo ultrasonic waves received by the ultrasonic receiver being less than the second reference value, turn off the air spray unit.

14. A control method of a clothes care apparatus, the control method comprising:
obtaining information regarding a target area at which contamination is located on clothes accommodated in a chamber of the clothes care apparatus;
moving, based on the information regarding the target area, at least one cleaning device, configured to remove the contamination, to a position at which the at least one cleaning device is capable of irradiating ultrasonic waves onto the target area;
controlling the at least one cleaning device to irradiate the ultrasonic waves onto the target area;
receiving echo ultrasonic waves reflected from the target area; and
controlling, based on a speed of the echo ultrasonic waves, a fan of the at least one cleaning device.

15. The control method of claim 14, wherein the controlling of the fan comprises:
based on the speed of the echo ultrasonic waves being equal to or greater than a first reference value, turning on the fan.

16. The control method of claim 15, further comprising controlling an air spray unit of the clothes care apparatus based on the speed of the echo ultrasonic waves.

17. The control method of claim 16, wherein the controlling of the fan comprises, based on the speed of the echo ultrasonic waves being less than a second reference value that is equal to or greater than the first reference value, turning on the fan.

18. The control method of claim 17, wherein the controlling the air spray unit comprises, based on the speed of the echo ultrasonic waves being less than the second reference value, turning off the air spray unit.

19. The control method of claim 18, wherein the controlling of the fan comprises, based on the speed of the echo ultrasonic waves received by the ultrasonic receiver being equal to or greater than the second reference value, turning on the fan.

20. The control method of claims 19, wherein the controlling of the air spray unit comprises, based on the speed of the echo ultrasonic waves received by the ultrasonic receiver being equal to or greater than the second reference value, turning on the air spray unit.
